(19) United States
(12) Patent Application Publication
(10) Pub. No.: US 2022/0354984 A1
(43) Pub. Date: Nov. 10, 2022

(54) PROCESS CHAMBER TO TREAT AIRBORNE CHEMICAL AND BIOLOGICAL CONTAMINATION

(71) Applicants: AMERICAN INNOVATIVE RESEARCH CORP., Bradford, NH (US); TCO SPOLKA Z OGRANICZONA ODPOWIEDZIALNOSCIA, Kraków (PL)

(72) Inventor: David W. PALMER, Bradford, NH (US)

(73) Assignees: AMERICAN INNOVATIVE RESEARCH CORP., Bradford, NH (US); TCO SPÓLKA Z OGRANICZONA ODPOWIEDZIALNOSCIA, Kraków (PL)

(21) Appl. No.: 17/638,491

(22) PCT Filed: Feb. 19, 2020
(86) PCT No.: PCT/IB2020/051367

§ 371 Date: Feb. 25, 2022

Related U.S. Application Data
(60) Provisional application No. 62/892,075, filed on Aug. 27, 2019.

(51) Int. Cl. A61L 9/20

(52) U.S. Cl. CPC A61L 9/205

(57) ABSTRACT

A self-cleaning air conduit for deactivating and decomposing chemical and life based airborne contamination. The conduit contains ultraviolet (UV) sources that irradiate semi UV reflective surfaces within the conduit. Said conduit is positioned in the path of pre and post filters. The internal surfaces of the conduit and components cast be altered by chemical and or mechanical means to maximize surfaces area and adhesion of photocatalytic coatings. Multiple coated panels within the conduit can be configured parallel to the air flow and positioned to enhancer the interaction of UV radiation with airborne contamination and photocatalytic produced free radical. Microprocessor based electronics connected to the conduit, provide power for key internal and ancillary componence necessary to control and monitor air velocity, UV radiation levels and airborne contamination levels. Pre and post filters can be positioned to retain VOC and deactivated life based airborne contamination and inert mineral based contamination.

### BACKGROUND

Numerous technologies have been implemented to reduce harmful airborne contamination responsible for creating or exacerbating respiratory complications, food supply spoilage, issues pertaining to farming of indoor crops, among other problems.

The choice of technologies varies depending on the perceived source of the contamination and the type of pathogens suspended in the air (airborne pollution).

The present disclosure contemplates an air cleaner in the form of an air conduit capable of self-cleaning the internal surfaces of the conduit. In a more limited aspect, the conduit uses ultraviolet radiation to sterilize airborne pathogens and fluid dynamics principles to enhance the photocatalytic decomposition of life forms and volatile organic compounds (VOC) into hydrocarbon based molecules.

### BRIEF DESCRIPTION OF THE DRAWINGS

The invention may take form in various components and arrangements of components, and in various steps and arrangements of steps. The drawings are only for purposes of illustrating preferred embodiments and are not to be construed as limiting the invention.

FIG. 1 is a partially exploded view of an air cleaning system according to a first exemplary embodiment.

FIG. 2 is an isometric view of a cell panel of the air cleaning system of FIG. 1, with an enlarged view of a modified panel surface having a titanium dioxide coating.

FIG. 3 is an illustration demonstrating fluid mechanic airflow at the surface of the panel of FIG. 2.

FIG. 4 is an isometric view of an electrical panel of the air cleaning system of FIG. 1.

FIG. 5 is an illustration demonstrating reduction of total volatile organic compounds by the air cleaning system of FIG. 1, at low, medium, and high air flows.

FIG. 6 is an isometric view of an air cleaning system in accordance with a second exemplary embodiment.

### DETAILED DESCRIPTION OF THE PREFERRED EMBODIMENTS

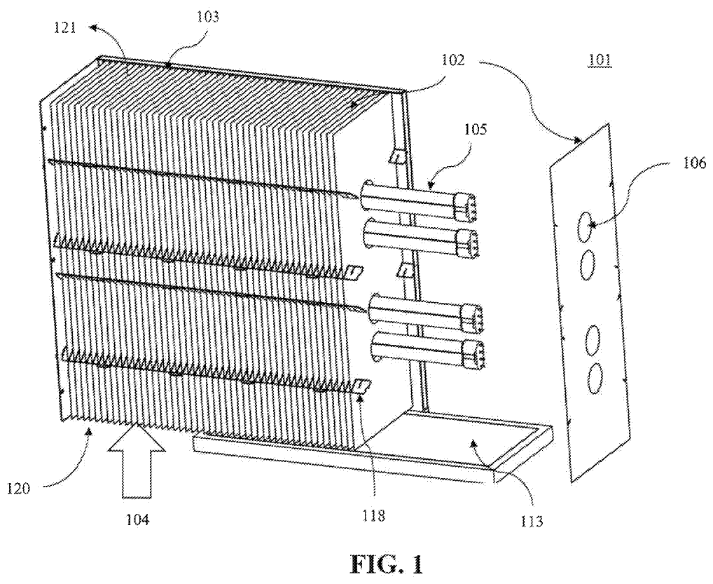

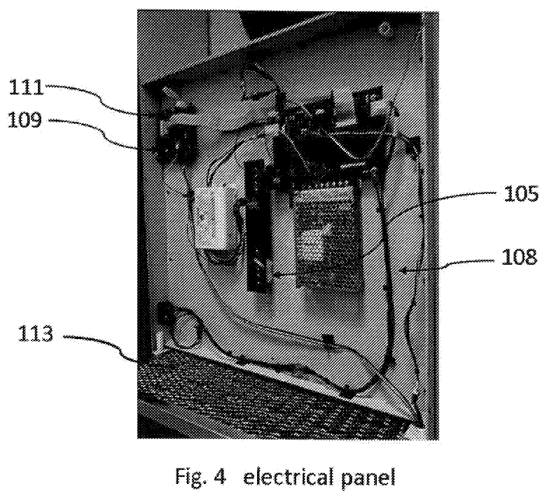

Referring now to the drawings, FIGS. 1-6 illustrate an air cleaning system for an air conduit. The air cleaning system, generally designated 101, includes a series of panels 102. In certain embodiments, the series of panels 102 substantially divide the system into a plurality of adjacent closed cells 103 and are configured to minimize resistance to air flow 104. In certain embodiments, the air flow 104 is directed into the housing through an inlet portion 120 of the air cleaning system and exits the housing through an outlet portion 121. In certain embodiments, each of the panels 102 has at least one opening 106 through which one or more UV lamps 105 are positioned.

In the exemplary preferred mechanical configuration appearing in FIG. 1, the air conduit 101 is divided by multiple parallel panels 102 to create a plurality of collinear closed cells 103 with minimum resistance to air flow 104.

In a preferred embodiment, the conduit internal wall and surfaces of multiple panels form narrow cells 103 enclosing multiple fluid paths between the conduit inlet 120 and conduit outlet 121. At least one ultraviolet (UV) lamp 105 is positioned to penetrate at least one opening 106 of the cell walls 102, perpendicular to both the air flow direction and cell walls. The radiation from the UV source or sources 105 has an unobstructed path to illuminate the volume of the cell 103 and cell surfaces 102.

In certain preferred embodiments, the surfaces of the panels 102 and the internal walls of the conduit 101 are mechanically or chemically altered to enhance the reflectivity of the UV radiation.

Exemplary means of altering the internal surface and panels 102 of the conduit 101 include, but are not limited to, sanding or chemical etching. In certain embodiments, panels 102 formed of a transparent or translucent material such as glass, plexiglass, or other transparent or translucent polymer material are used to allow UV radiation to be shared by adjacent cells to thereby enhance the effectiveness of the UV radiation.

Also, in certain embodiments, edge-lit translucent panels are employed to allow UV radiation to be shared by adjacent cells to enhance the effectiveness of the UV activated photocatalytic surfaces by providing energy within the photocatalytic coating.

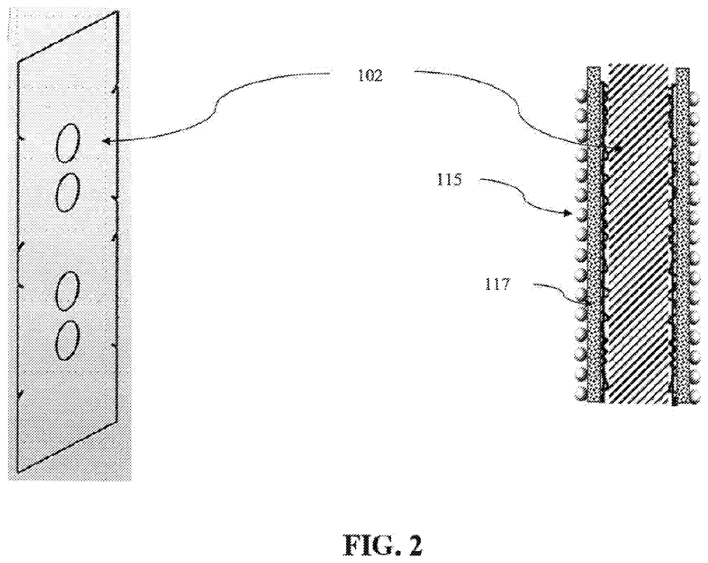

In certain preferred embodiments, openings 106 have been cut through all the panels to allow for positioning of two UV sources 105. Exemplary preferred UV sources include but are not limited to UVC lamps and UVA lamps. In certain embodiments, multiple UV sources are included. In certain further embodiments, the inclusion of multiple UV sources may be configured for use in an alternating, on-off sequence to extend the total useful life for UV exposure within the self-cleaning conduit. Alternative UV sources could be diodes selected for similar UVC and UVA wavelengths. In an exemplary alternating on-off sequence, the first lamp runs for 2 years then turns off, the second lamp is turned on for 2 years and both lamps are then turned on for an additional 2 years. Such alternating/intermittent use may extend the life expectancy of the system utilizing UV sources from 2 years to 6 years.
Germicidal UV Radiation: The deactivation of life viable airborne contaminants occurs in the interstitial space 103 between the cell walls 102. UV direct and reflected radiation has been proven to damage (inactivate) the DNA in the most common airborne organic pathogens at high UV radiation intensities and exposure time (doses). In a preferred embodiment, the panels 102 and conduit 101 are constructed of highly UV reflective aluminum having one or more altered surfaces 116 (see FIG. 2) of the panel 102 to amplify the reflective dose of the UV source, and to ensure that airborne pathogens moving through the system 100 and cells are irradiated multiple times from multiple directions. Most UV radiation loss occurs through the inlet 120 and outlet 121. The lost radiation helps to clean an optional pre-filter 113 (such as a carbon filter, e.g., an activated carbon filter). The lost UV radiation also provides thermal energy to increase the absorption capability of the filter 113, such as an activated carbon filter, if employed. The preferred UV radiation for airborne pathogens is UVC radiation.
UVA: In certain embodiments, UVA radiation is utilized in combination with a titanium dioxide coating surface on the panels 102. When airborne moisture contacts a TiO2. surface activated by UVA radiation, a catalytic reaction converts H2O into free radicals 115 that deconstruct hydrocarbon molecules into CO2 and water. The beneficial interaction occurs at the interface of the TiO2 surface 117 and ambient airborne moisture.

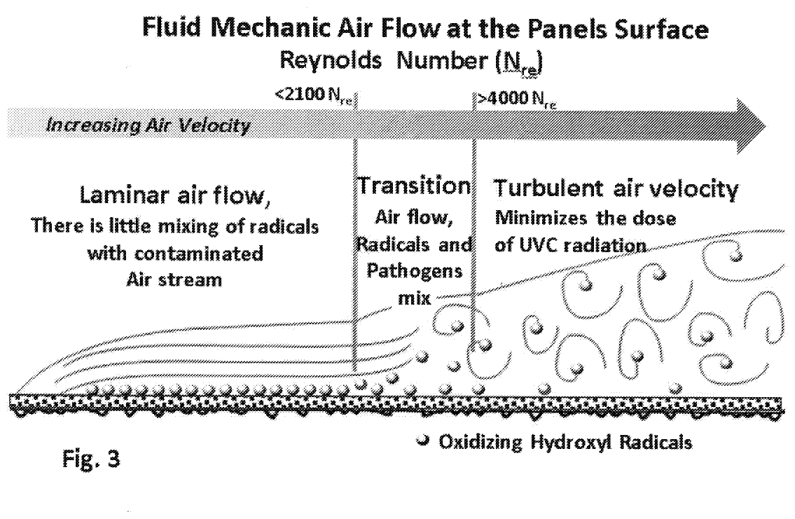

In certain preferred embodiments, a transparent TiO2 suspended in a liquid solution is applied to the modified aluminum surface 107 and allowed to dry. The modified aluminum surface 107 provides an enhanced TiOz bond and greater surface area. In certain embodiments, the reflected UVA from the larger modified aluminum substrate 107 doubles the TiO2 catalytic free radical yield. The modified aluminum surface 107 also enables a thicker more durable photocatalytic self-cleaning coating. FIG. 2 illustrates the boundary layer 115 between the air flow 104 and the titanium dioxide coating 117 of a panel 102.
Air Stream Velocity: The velocity of the polluted air stream plays an important part in the air decontamination process. The UVC radiation dose a pathogen receives is inversely proportional to the air velocity (resident time in the conduit) and directly proportional to the proximity of the UV source. As the air velocity is increased (to high), the effect of the UVC radiation on DNA deactivation decreases. If the air velocity is decreased (to low) within the conduit, alaminar air flow will develop, allowing a pathogen to follow through in between panels 102, forming streamlines and passing through the conduit without making contact with the TiO₂—H₂O free radical boundary layer 115 (see FIG. 3).

The photocatalytic process occurs at the boundary layer 115 of the air stream 104 and the TiO₂ coating 117. The boundary layer 115 at the surface of the TiO₂ 117 coating will contain the richest concentration of free radicals. Therefore, to ensure that the pathogens come in contact with free radicals, an air stream with a minimum Reynolds Number (Nre) above 2100 (transition air flow) should be maintained (see FIG. 3).

In a simplified design, a target of approximately 3500 Nre may be maintained.

In certain preferred embodiments, a lower Nre may be utilized by adding mechanical turbulence generators 118 may be added at the edges of the cells to lower the required velocity, ensuring transition from laminar to turbulent air flow at a lower velocity. In a preferred embodiment, the air is aggressively disturbed by one or more generator 118 and transitions to a mixed flow. There are numerous aerodynamic methods usable to initiate instability at low air velocities<Nre 2 00. Air turbulence shed from fan blades, spoilers, and vortex generators, to name a few. Disclosed is a configuration that adds air obstructions 118 with in the cell 103 walls to create different air velocities within the same cell, triggering instability. The air velocity is created and maintained by at least one variable controlled fan.

### VOC Reduction VS. Air Flow Instability

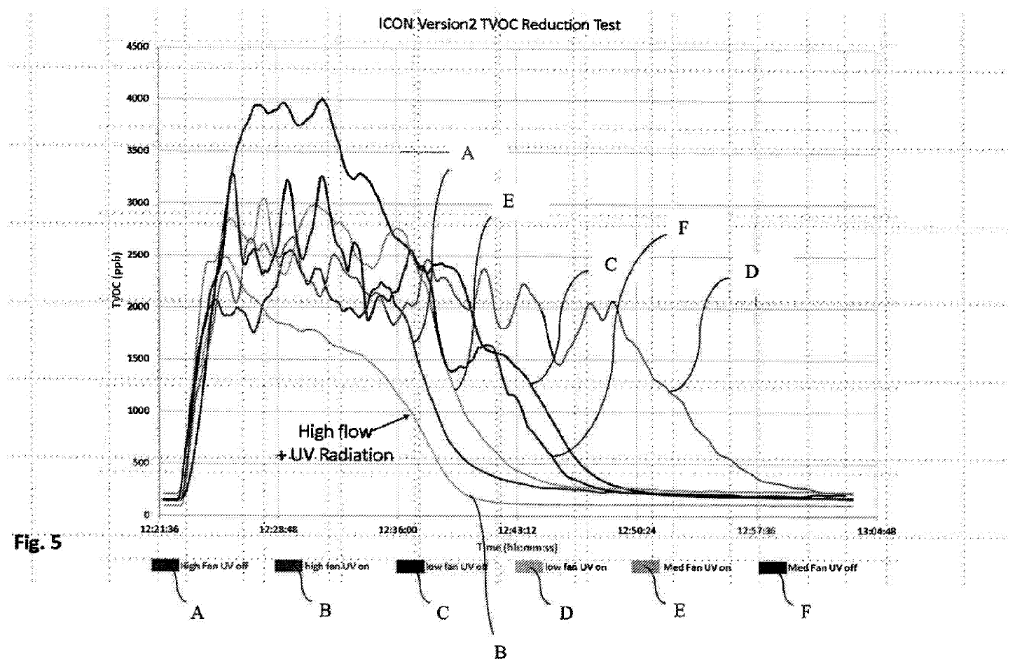

In certain preferred embodiments, the reduction of total VOC (TVOC) airborne contamination is accomplished within the preferred conduit configuration at higher air flow transition states (see FIG. 5) with mechanically stimulated air instability. FIG. 5 illustrates exemplary reduction of TVOC at low, medium and high air flows. All three airflows conditions have been chosen to be within conventional Reynolds Transition Numbers (Nre) enhanced by turbulent generation. It is evident that the higher the air velocity, the higher the TVOC reduction. This is counterintuitive to all other air cleaning devices.

### In a Second Conduit Embodiment

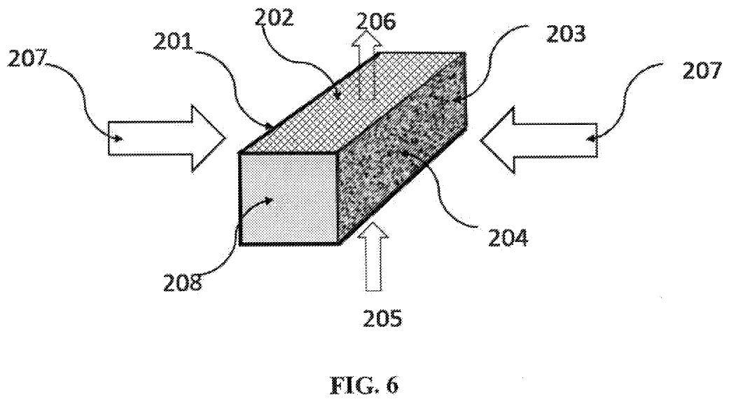

Disclosed is a self-cleaning single cell 201 defined by two solid opposing UV transparent side walls 203 and two opposing solid, UV reflective end caps 208. The end caps surfaces can be mechanically or chemically altered to increase surface area and UV reflectivity. As illustrated in FIG. 6, the top and bottom of the cell 201 are enclosed by two opposing air permeable grid structures 202 that enable air to flow into the cell through inlet 205 and air to flow out of the cell through outlet 206. The two opposing air permeable grid structures 202 also retains the aggregate that fills the cell 201.

The air permeable aggregate 204 is selected to be UV 207 transparent and can be in one or more of the following granular configurations: spheres, pellets, chips and/or flakes.

After assembling the cell 201, all surfaces of the cell 201 and aggregate 204 may be coated with TiO₂, by submerging the assembly 201 and aggregate 204 in a liquid containing TiO₂ and lefl to dry.

A fan, not shown, moves air through the aggregate 204 from inlet 205 to outlet 206 exposing the air to at least one wavelength of UV light directed at the transparent side walls 203. The UV source 207 for illuminating the transparent aggregate 204 may include UV lamps, UV LEDs, or both. In the second embodiment, the UV source 207 may be, but is not limited to UVA and/or UVC sources. The UVC energy dispersed throughout the aggregate 204 disrupts the DNA of airborne pathogens, disabling the replication of the pathogen. The UVA energy dispersed thought the aggregate 204 activates a Photocatalytic reaction on all internal surfaces of the cell 201 and all surfaces of the TiO₂ coated aggregate 204. The TiO₂ reacts with H₂O in the air, creating free radicals that break down volatile organic compounds (VOC) and decompose all organic debris disposed on the internal cell 201 and aggregate 204 surfaces.

### External Electronics

In a preferred embodiment, external electronics panel 108 is rigidly attached to the outside of the conduit, to power the UV lamps 105, UV sensors (109 not shown), air flow sensors and airborne contamination sensors (111 not shown). In a preferred embodiment, the electronics would be capable of controlling an upstream or downstream fan. Also the electronics would have the capability communicate with other electronic systems, facilities by wire, RFC and or internet, providing real time monitoring of the air cleaning process.

### External Filters

Replaceable activated carbon pre filters 113 provided at the input 120, and/or output (not shown) of the conduit evenly distributes the air flow through the cells. The carbon remove airborne volatile compounds (VOC) and before and after becoming saturated, provide an optical shield to prevent downstream and upstream escape of UV radiation. UVC &UVA radiation illuminating the activated carbon will clean carbon surfaces, of both pre and post to enable additional VOC airborne pathogen reduction.

The invention has been described with reference to the preferred embodiment. Modifications and alterations will occur to others upon a reading and understanding of the preceding detailed description. It is intended that the invention be construed as including all such modifications and alterations.

1.-28 (canceled)

29. An air conduit system, comprising a housing with an air inlet connected to an air outlet to create a contained airflow path and a multiple cells defined by surfaced textured panels, which are parallel to the direction of the contained airflow, each surface of every cell is coated with a UV transparent photocatalytic layer, where the air conduit system further comprises at least one UV source, characterized in that the air conduit system comprises plurality of the air obstruction elements that are provided at the edges of the cells to create different air velocities within the cell for generating a transition phase airflow.

30. The air conduit system according to claim 29, wherein the air obstruction elements are mechanical turbulence elements such as fan blades, spoilers or vortex generators.

31. The air conduit system according to claim 29, wherein all internal surfaces are mechanically or chemically textured.

32. The air conduit system according to claim 31, wherein all internal surfaces are textured by sanding or chemical etching.

33. The air conduit system according to claim 29, wherein panels are formed of transparent or translucent material such as glass, plexiglass or polymer material.

34. The air conduit system according to claim 29, wherein textured panels on their surfaces are coated with TiO₂.

35. The air conduit system according to claim 29, wherein the panels comprise at least one opening through which at least one UV source is positioned.

36. The air conduit system according to claim 29, comprising at least two UV sources with at least one UVC lamp and at least one UVA lamp.

37. The air conduit system according to claim 29, wherein the air velocity in the cells is maintained by a computer controlled fan.

38. An air conduit system comprising a housing in a form of a single cell, having two solid opposing UV transparent side walls and two opposing solid, UV reflective end caps which are surfaced textured, where the top and bottom of the cell is enclosed by two opposing permeable grid structures to enable air to flow into the cell through inlet and air to flow out of the cell through outlet, where the air conduit system further comprises at least one UV source, which is located outside the conduit, characterized in that inside the cell the volume of the conduit is Idled with contiguous photocatalytic coated UV transparent aggregate to create different air velocities within the cell for generating a transition phase airflow.

39. The air conduit system according to claim 38, wherein the volume of the conduit is filled with an aggregate consisting of contiguous photoactinic coated UV transparent particles in a form of spheres or pellets or chips or flakes.

40. The air conduit system according to claim 38. wherein end cap surfaces are mechanically or chemically textured.

41. The air conduit system according to claim 40, wherein end cap surfaces are textured by sanding or chemical etching.

42. The air conduit system according to claim 38, wherein end cap surfaces are coated with TiO₂.

43. The air conduit system according to claim 38, wherein at least one UV source can be UVC lamp or UVA lamp.

44. The air conduit system according to claim 38, wherein the air velocity in the cell is maintained by a computer controlled fan.

45. The air conduit system according to claim 38, wherein the conduit is free of all electronics.
